(19) United States
(12) Patent Application Publication
(10) Pub. No.: US 2011/0112101 A1
(43) Pub. Date: May 12, 2011

(54) TREATMENT FOR OCULAR-RELATED DISORDERS

(75) Inventors: Ramalinga DHARANIPRAGADA, Belle Mead, NJ (US); Thomas OLIGINO, Forks Township, PA (US); Chang S. HAHN, Princeton, NJ (US); Timothy A. GILLESPY, Hillsborough, NJ (US); Ellen MATZKIN, Bridgewater, NJ (US)

(73) Assignee: SANOFI-AVENTIS, Paris (FR)

(21) Appl. No.: 12/879,232

(22) Filed: Sep. 10, 2010

Related U.S. Application Data
(63) Continuation of application No. PCT/US2009/036119, filed on Mar. 5, 2009.

(51) Int. Cl. A61K 31/4985; A61P 27/02

(52) U.S. Cl. 514/249

(57) ABSTRACT

A treatment for ocular related disorders, such as macular degeneration, and preferably age-related macular degeneration, diabetic retinopathy, and diabetic macular edema is disclosed, using a compound of Formula I.

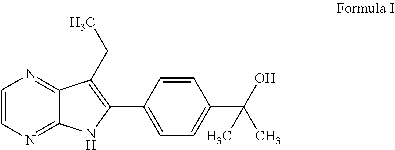

This application is a continuation of International Application No. PCT/US2009/036119, filed Mar. 5, 2009, which is incorporated herein by reference in its entirety.

### FIELD OF THE INVENTION

This invention is directed to methods of therapy for human and non-human patients suffering from, or subject to, ocular disorders such as age related macular degeneration, diabetic retinopathy, and diabetic macular edema.

### BACKGROUND OF THE INVENTION

Protein kinases participate in the signalling events which control the activation, growth and differentiation of cells in response to extracellular mediators and to changes in the environment. In general, these kinases fall into several groups; those which preferentially phosphorylate serine and/or threonine residues and those which preferentially phosphorylate tyrosine residues [S. K. Hanks and T. Hunter, FASEB. J., 1995, 9, pages 576-596]. The serine/threonine kinases include for example, protein kinase C isoforms [A. C. Newton, J. Biol. Chem., 1995, 270, pages 28495-28498] and a group of cyclin-dependent kinases such as cdc2 [J. Pines, Trends in Biochemical Sciences, 1993, 18, pages 195-197]. The tyrosine kinases include membrane-spanning growth factor receptors such as the epidermal growth factor receptor [S. Iwashita and M. Kobayashi, Cellular Signalling, 1992, 4, pages 123-132], and cytosolic non-receptor kinases such as p56tck, p59fYn, ZAP-70 and csk kinases [C. Chan et. al., Ann. Rev. Immunol., 1994, 12, pages 555-592]. Inappropriately high protein kinase activity has been implicated in many diseases resulting from abnormal cellular function. This might arise either directly or indirectly, for example by failure of the proper control mechanisms for the kinase, related for example to mutation, over-expression or inappropriate activation of the enzyme; or by over- or underproduction of cytokines or growth factors also participating in the transduction of signals upstream or downstream of the kinase. In all of these instances, selective inhibition of the action of the kinase might be expected to have a beneficial effect.

Syk is a 72-kDa cytoplasmic protein tyrosine kinase that is expressed in a variety of hematopoietic cells and is an essential element in several cascades that couple antigen receptors to cellular responses. Thus, Syk plays a pivotal role in signalling of the high affinity IgE receptor, FcεR1, in mast cells and in receptor antigen signalling in T and B lymphocytes. The signal transduction pathways present in mast, T and B cells have common features. The ligand binding domain of the receptor lacks intrinsic tyrosine kinase activity. However, they interact with transducing subunits that contain immunoreceptor tyrosine based activation motifs (ITAMs) [M. Reth, Nature, 1989, 338, pages 383-384]. These motifs are present in both the α and γ subunits of the FcεR1, in the ξ-subunit of the T cell receptor (TCR) and in the IgGα and IgG γ subunits of the B cell receptor (BCR). [N. S. van Oers and A. Weiss, Seminars in Immunology, 1995, 7, pages 227-236]. Upon binding of antigen and multimerization, the ITAM residues are phosphorylated by protein tyrosine kinases of the Src family. Syk belongs to a unique class of tyrosine kinases that have two tandem Src homology 2 (SH2) domains and a C terminal catalytic domain. These SH2 domains bind with high affinity to ITAMs, and this SH2-mediated association of Syk with an activated receptor stimulates Syk kinase activity and localises Syk to the plasma membrane.

Angiogenesis or the formation of new blood vessels by sprouting from the preexisting vasculature is of central importance for embryonic development and organogenesis. Abnormal enhanced neovascularization is observed in rheumatoid arthritis, diabetic retinopathy and during tumor development (Folkman, Nat. Med., 1995, 1, 27-31.). Angiogenesis is a complex multistage process which includes activation, migration, proliferation and survival of endothelial cells. Extensive studies in the field of tumor angiogenesis in the past two decades have identified a number of therapeutic targets including kinases, proteases and integrins resulting in the discovery of many new anti-angiogenic agents, including KDR inhibitors some of which are currently under clinical evaluation (Jekunen, et al Cancer Treatment Rev. 1997, 23, 263-286.). Angiogenesis inhibitors may be used in frontline, adjuvant and even preventive settings for the emergence or regrowth of malignancies.

Important causes of severe vision loss and blindness are ocular-related disorders where the vascular system of the eye is damaged or insufficiently regulated. Ocular-related diseases comprising a neovascularization aspect are many and include, for example, age-related macular degeneration, diabetic retinopathy, and diabetic macular edema. It is likely that severe vision loss does not result directly from neovascularization, but from the effect of neovascularization on the retina. The retina is a delicate ocular membrane on which images are received. Near the center of the retina is the macula lutea, an oval area of retinal tissue where visual sense is most acute. The retina is most delicate at the fovea centralis, the central depression located in the center of the macula. Damage of the retina such as retinal degeneration is directly connected to vision loss. While a common cause of retinal detachment, retinal tears, and retinal degeneration is abnormal, i.e., uncontrolled, vascularization of various ocular tissues, this is not always the case. Atrophic complications associated with age-related macular degeneration, nonproliferative diabetic retinopathy, and inflammatory ocular damage are not associated with neovascularization, but can result in severe vision loss if not treated. Disorders associated with both neovascular and atrophic components, such as age-related macular degeneration, diabetic retinopathy and diabetic macular edema, are particularly difficult to treat due to the emergence of a wide variety of complications. See Diabetic Retinopathy: A Review; June Chu and Yusuf Ali; Drug Development Research 69: 1-14 (2008); New Developments in Diabetic Retinopathy; Expert Rev. Ophthalmol. 2(6), 947-956 (2007); and Diabetic Retinopathy; Robert Frank; N. Engl. J. Med. 350; 1; 48-58 (2004).

What has been discovered now is in one embodiment, a method of prophylactically or therapeutically treating a patient for an ocular related disorder, wherein the method comprises administering to the patient a pharmaceutically effective amount of a compound of formula I, or a pharmaceutically acceptable salt or prodrug thereof. Administration to an individual at risk of abnormal angiogenesis or vascularization of the eye can prevent abnormal angiogenesis of the eye, especially the retina. The patient is at risk of, or has been diagnosed with, abnormal angiogenesis of the eye, and is transformed by treatment with the compound of the invention from a disease susceptible state to a disease resistant state. Untreated, this type of disease is characterized by invasion of new blood vessels into the structures of the eye, such as the retina. It is a common cause of blindness and is involved in approximately twenty eye diseases. In age-related macular degeneration, the associated visual problems are caused by an ingrowth of choroidal capillaries through defects in Bruch's membrane with proliferation of fibrovascular tissue beneath the retinal pigment epithelium. Angiogenic damage is also associated with diabetic retinopathy and diabetic macular edema.

Age-related macular degeneration (AMD) is a degenerative condition of the macula (the central retina). It is the most common cause of vision loss in the United States in those 50 or older, and its prevalence increases with age. AMD is caused by hardening of the arteries that nourish the retina. This deprives the sensitive retinal tissue of oxygen and nutrients that it needs to function and thrive. As a result, the central vision deteriorates.

Macular degeneration varies widely in severity. In the worst cases, it causes a complete loss of central vision, making reading or driving impossible. For others, it may only cause slight distortion. Fortunately, macular degeneration does not cause total blindness since it does not affect the peripheral vision.

AMD is classified as either wet (neovascular) or dry (non-neovascular). About 10% of patients who suffer from macular degeneration have wet AMD. This type occurs when new vessels form to improve the blood supply to oxygen-deprived retinal tissue. However, the new vessels are very delicate and break easily, causing bleeding and damage to surrounding tissue. While wet AMD represents a significant minority of AMD cases, it is associated with 90% of significant vision loss associated with the disorder.

Current treatments for AMD are focused on affecting the neovascularization that leads to the vision loss associated with wet AMD. The current gold standard therapies are biologicals that are injected directly into the eye. These are listed in Table 1. These treatments are expensive, cumbersome and limited by dose. There is a clear need for effective oral therapies for this disorder.

TABLE 1

| | | MODE OF | ROUTE OF |
|---|---|---|---|
| AGENT | COMPANY | ACTION | ADMINISTRATION |
| Laser | Multiple | Laser mediated | Laser |
| | | vessel occlusion | |
| Visudyne | Novartis | Reactive oxygen | Intravenous then laser |
| | | radicals | activation |
| | | mediated vessel | |
| | | occlusion | |
| Macugen | EyeTech/OSI | Anti-VEGF | Intravitreal |
| | | inhibitor (RNA) | |
| Lucentis | Genentech | Anti-VEGF | Intravitreal |
| | Novartis | mAb | |

In view of the current situation regarding therapies for treating macular degeneration, it is clear that there is a need for more effective and better tolerated therapies.

Diabetic retinopathy is an ocular disorder that is subdivided into two stages. These are a non-proliferative stage, usually occurring first, and a proliferative stage. Non-proliferative diabetic retinopathy frequently leads to vision loss in patients due to retinal edema, in particular diabetic macular edema, this resulting in vascular leakage. Focal and diffuse macular leakage occurs as a result of microvascular abnormalities, intraretinal microaneurysms, capillary closure, and retinal hemorrhages. Prolonged periods of vascular leakage ultimately lead to thickening of the basement membrane and formation of soft and hard exudates. Non-proliferative diabetic retinopathy is also characterized by loss of retinal pericytes. The proliferative stage of diabetic retinopathy is characterized by neovascularization and fibrovascular growth (i.e., scarring involving glial and fibrous elements) from the retina or optic nerve over the inner surface of the retina or disc or into the vitreous cavity. Retinal neovascularization is the leading cause of vision loss associated with proliferative diabetic retinopathy.

Current approaches to the treatment of abnormal angiogenesis and vascularization in the eye include laser therapy, which destroys some retinal tissue in order to preserve some vision. There remains a clear need for improved methods and agents for prevention and treatment of conditions involving abnormal angiogenesis and harmful angiogenesis such as pathological angiogenesis in the tissues of the eye.

Given the prevalence of ocular-related disorders, there remains a need for an effective prophylactic and therapeutic treatment of ocular-related disorders, in particular those ocular-related disorders associated with both atrophic and neovascular complications, such as age-related macular degeneration, diabetic retinopathy, and diabetic macular edema. Accordingly, the present invention is concerned with a compound and methods for prophylactically and therapeutically treating ocular-related disorders. This and other advantages of the present invention will become apparent from the detailed description provided herein.

### SUMMARY OF THE INVENTION

The present invention relates to a method of treating macular degeneration, and more specifically age-related macular degeneration using a compound of Formula I:

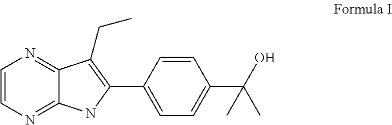

This invention is directed to a substituted azaindole of Formula I, which has now been found to be active in the inhibition of macular degeneration in animal models.

Another aspect of the present invention is a treatment for wet AMD.

The compound of Formula I may be used as an effective oral treatment for AMD. Furthermore we envision, based on our data, that Syk inhibitors in general can be useful agents for the treatment of this disorder. Data for these conclusions is outlined below.

Another aspect of the present invention is a method of treating diabetic retinopathy by administering to a patient in need thereof, a pharmaceutically effective amount of a compound of Formula I.

Yet another aspect of the present invention is a method of treating diabetic macular edema by administering to a patient in need thereof, a pharmaceutically effective amount of a compound of Formula I.

Yet another aspect of the present invention is a treatment for macular degeneration, diabetic retinopathy or diabetic macular edema by treating a patient with a Syk inhibitor in general.

### DETAILED DESCRIPTION OF THE INVENTION

Thus, in one aspect, the present invention is directed to therapeutic treatments and pharmaceutical compositions comprising a compound of general formula (I):

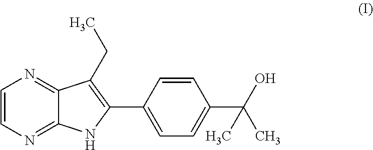

which also may be known as: 2-[4-(7-Ethyl-5H-pyrrolo[2,3-b]pyrazin-6-yl)-phenyl]-propan-2-ol. The synthesis of this compound is known to a person skilled in the art from its publication in International Application WO 2008/033798. It is also known from this publication that this compound is a Syk inhibitor.

In the present specification, the term “compound of the invention”, and equivalent expressions, are meant to embrace a compound of general formula (I) as hereinbefore described, which expression includes the prodrugs, the pharmaceutically acceptable salts, and the solvates, e.g. hydrates, where the context so permits. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

### DEFINITIONS

As used above, and throughout the description of the invention, the following terms, unless otherwise indicated, shall be understood to have the following meanings:

“Patient” includes both human and other mammals.

“Pharmaceutically acceptable ester” refers to esters that hydrolyze in vivo and include those that break down readily in the human body to leave the parent compound or a salt thereof, Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Exemplary esters include formates, acetates, propionates, butyrates, acrylates, ethylsuccinates, and the like.

“Pharmaceutically acceptable prodrugs” as used herein refers to those prodrugs of the compounds of the present invention which are, within the scope of sound medical judgment, suitable for use in contact with the tissues of patients with undue toxicity, irritation, allergic response, and the like, commensurate with a reasonable benefit/risk ratio, and effective for their intended use of the compounds of the invention. The term “prodrug” refers to compounds that are rapidly transformed in vivo to yield the parent compound of the above formula, for example by hydrolysis in blood. Functional groups that may be rapidly transformed, by metabolic cleavage, in vivo form a class of groups reactive with the carboxyl group of the compounds of this invention. They include, but are not limited to such groups as alkanoyl (such as acetyl, propanoyl, butanoyl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds of this invention are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion is provided in Design of Prodrugs, H. Bundgaard, ed., Elsevier (1985); Methods in Enzymology; K. Widder et al, Ed., Academic Press, 42, 309-396 (1985); A Textbook of Drug Design and Development, Krogsgaard-Larsen and H. Bandaged, ed., Chapter 5; “Design and Applications of Prodrugs” 113-191 (1991); Advanced Drug Delivery Reviews, H. Bundgard, 8, 1-38, (1992); J. Pharm. Sci., 77., 285 (1988); Chem. Pharm. Bull., N. Nakeya et aI, 32, 692 (1984); Pro-drugs as Novel Delivery Systems, T. Higuchi and V. Stella, 14 A.C.S. Symposium Series, and Bioreversible Carriers in Drug Design, E. B. Roche, ed., American Pharmaceutical Association and Pergamon Press, 1987, which are incorporated herein by reference.

“Pharmaceutically acceptable salts” refers to the relatively non-toxic, inorganic and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-B-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977). Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

### EMBODIMENTS

With reference to inventions described herein, below are particular embodiments related thereto.

A particular embodiment according to the invention is the treatment of macular degeneration by administering to a patient in need thereof, a pharmaceutically effective amount of a compound of Formula I.

Another particular embodiment according to the invention is the treatment of macular degeneration by administering to a patient in need thereof, a pharmaceutically effective amount of a Syk inhibitor.

A preferred embodiment according to the invention is where the macular degeneration is age-related macular degeneration and the effective amount is of a compound of Formula I.

Another preferred embodiment according to the invention is where the macular degeneration is age-related macular degeneration and the effective amount is of a Syk inhibitor.

Another embodiment according to the invention is the treatment of diabetic retinopathy by administering to a patient in need thereof, a pharmaceutically effective amount of a compound of Formula I.

Another embodiment according to the invention is the treatment of diabetic macular edema by administering to a patient in need thereof, a pharmaceutically effective amount of a compound of Formula I.

The compounds of the invention optionally are supplied as salts. Those salts that are pharmaceutically acceptable are of particular interest since they are useful in administering the foregoing compounds for medical purposes. Salts that are not pharmaceutically acceptable are useful in manufacturing processes, for isolation and purification purposes, and in some instances, for use in separating stereoisomeric forms of the compounds of this invention. The latter is particularly true of amine salts prepared from optically active amines.

An aspect of the present invention is to provide a pharmaceutical composition comprising, a pharmaceutically effective amount of a compound of formula I and pharmaceutically acceptable carrier or diluent.

Another aspect of the invention to provide a pharmaceutical composition which is effective, in and of itself, for utilization in a beneficial combination therapy because it includes a plurality of active ingredients which may be utilized in accordance with the invention.

The amount of the compound of Formula I in any of the foregoing applications can be a pharmaceutically effective amount, a suboptimal effective amount, or combinations thereof, so long as the final combination of ingredients comprises a pharmaceutically effective amount of compounds that is effective in treating or preventing macular degeneration, diabetic retinopathy, or diabetic macular edema in a patient.

### In-Vivo Efficacy of the Compound of Formula I in Models of Ocular-Related Disorders Such as AMD

Several models of wet AMD are available in the rodent and rabbit. The pre-clinical efficacy of the compound of Formula I was assessed in a laser induced model of macular degeneration in the rat. In this model, injury is induced on the retina of the rat by a focused laser beam. This injury results in vascularization and inflammation, and the subsequent formation of a plaque on the retina that is associated with loss of vision. The ability of treatments with a compound of Formula Ito inhibit the size and thickness of such plaques serves as an indicator for the potential for the treatment of wet AMD patients. A krypton laser was used to irradiate the retina of a rat eye at 75 microns for 0.1 seconds, at each of 3-4 spots. From days 1 to 14, the irradiated rats were dosed twice a day with the compound of Formula I perorally (bid PO). Doses used were 3 mg/kg, 10 mg/kg, and 30 mg/kg body weight. An additional treatment group was given the vehicle for the oral compound, also bid PO administration. A positive control was used with 0.5 mg triamcinolone delivered to the intraocular subtendon. At 14 days after such irradiation, the rat was perfused with FITC-dextran (fluorescein isothiocyanate dextran) and the eyes were removed for evaluation. Evaluation was by fundoscopy and fluorescein angiography. The data with the compound of Formula I in this model demonstrated that the compound significantly reduced plaque thickness at 30 and 10 mg/kg bid PO. These results were significantly better than what was observed with the positive control triamcinolone (intravitreal injection of a steroid). The results are shown in Table 2.

TABLE 2

| | % Inhibition of | |
|---|---|---|
| Treatment | Plaque | Significance |
| Vehicle | 0 | |
| 3 mg/kg compound of Formula I | 12 | |
| 10 mg/kg compound of Formula I | 36 | p < 0.003 |
| 30 mg/kg compound of Formula I | 33 | p < 0.0006 |
| 0.5 mg triamcinolone | 26 | p < 0.0245 |

Another aspect of the present invention herein described is a method of treating a patient suffering from or subject to macular degeneration, comprising administering to the patient a pharmaceutically effective amount of a Syk inhibitor. References herein to “treating” should be understood to include prophylactic therapy to prevent or inhibit a disorder, as well as the treatment of a patent to ameliorate or improve a disorder that the patient has. “Effective amount” is meant to describe an amount of the compound of the present invention effective within the scope of reasonable biological judgment, suitable for use in contact with the cells of humans and other mammals without undue toxicity, irritation, allergic response and the like, and are commensurate with a reasonable benefit/risk ratio in treating and thus producing the desired therapeutic effect.

A particular aspect of the invention provides for a compound according to the invention to be administered in the form of a pharmaceutical composition, though the compound may be administered alone. “Pharmaceutical composition” means a composition comprising a compound of formula I and at least one component selected from the group comprising pharmaceutically acceptable carriers, diluents, coatings, adjuvants, excipients, or vehicles, such as preserving agents, fillers, disintegrating agents, wetting agents, emulsifying agents, emulsion stabilizing agents, suspending agents, isotonic agents, sweetening agents, flavoring agents, perfuming agents, coloring agents, antibacterial agents, antifungal agents, other therapeutic agents, lubricating agents, adsorption delaying or promoting agents, and dispensing agents, depending on the nature of the mode of administration and dosage forms. The compositions may be presented in the form of tablets, pills, granules, powders, aqueous solutions or suspensions, injectable solutions, elixirs or syrups. Exemplary suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Exemplary antibacterial and antifungal agents for the prevention of the action of microorganisms include parabens, chlorobutanol, phenol, sorbic acid, and the like. Exemplary isotonic agents include sugars, sodium chloride and the like. Exemplary adsorption delaying agents to prolong absorption include aluminum monostearate and gelatin. Exemplary adsorption promoting agents to enhance absorption include dimethyl sulfoxide and related analogs. Exemplary carriers, diluents, solvents, vehicles, solubilizing agents, emulsifiers and emulsion stabilizers, include water, chloroform, sucrose, ethanol, isopropyl alcohol, ethyl carbonate, ethyl acetate, benzyl alcohol, tetrahydrofurfuryl alcohol, benzyl benzoate, polyols, propylene glycol, 1,3-butylene glycol, glycerol, polyethylene glycols, dimethylformamide, Tween® 60, Span® 60, cetostearyl alcohol, myristyl alcohol, glyceryl mono-stearate and sodium lauryl sulfate, fatty acid esters of sorbitan, vegetable oils (such as cottonseed oil, groundnut oil, com germ oil, olive oil, castor oil and sesame oil) and injectable organic esters such as ethyl oleate, and the like, or suitable mixtures of these substances. Exemplary excipients include lactose, milk sugar, sodium citrate, calcium carbonate, dicalcium phosphate. Exemplary disintegrating agents include starch, alginic acids and certain complex silicates. Exemplary lubricants include magnesium stearate, sodium lauryl sulfate, talc, as well as high molecular weight polyethylene glycols.

Other therapeutic agents may be used in combination with a compound of the present invention, including other anti agents. Therapeutic agents used in combination with a compound of the present invention may be administered separately, simultaneously or sequentially. The choice of material in the pharmaceutical composition other than the compound of formula I is generally determined in accordance with the chemical properties of the active compound such as solubility, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulfate and talc may be used for preparing tablets.

The pharmaceutical compositions may be presented in assorted forms such as tablets, pills, granules, powders, aqueous solutions or suspensions, injectable solutions, elixirs or syrups.

“Liquid dosage form” means the dose of the active compound to be administered to the patient is in liquid form, for, example, pharmaceutically acceptable emulsions, solutions, suspensions, syrups and elixirs. In addition to the active compounds, the liquid dosage forms may contain inert diluents commonly used in the art, such solvents, solubilizing agents and emulsifiers.

Solid compositions may also be employed as fillers in soft and hard-filled gelatin capsules using such excipients as lactose or milk sugar as well as high molecular weight polyethylene glycols, and the like.

When aqueous suspensions are used they can contain emulsifying agents or agents which facilitate suspension.

The oily phase of the emulsion pharmaceutical composition may be constituted from known ingredients in a known manner. While the phase may comprise merely an emulsifier (otherwise known as an emulgent), it desirably comprises a mixture of at least one emulsifier with a fat or an oil or with both a fat and an oil. In a particular embodiment, a hydrophilic emulsifier is included together with a lipophilic emulsifier that acts as a stabilizer. Together, the emulsifier(s) with or without stabilizer(s) make up the emulsifying wax, and the way together with the oil and fat make up the emulsifying ointment base which forms the oily dispersed phase of the cream formulations.

If desired, the aqueous phase of the cream base may include, for example, a least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound that enhances absorption or penetration of the active ingredient through the skin or other affected areas.

The choice of suitable oils or fats for a formulation is based on achieving the desired properties. Thus the cream should preferably be a non-greasy, non-staining and washable product with suitable consistency to avoid leakage from tubes or other containers. Straight or branched chain, mono- or dibasic alkyl esters such as di-isopropyl myristate, decyl oleate, isopropyl palmitate, butyl stearate, 2-ethylhexyl palmitate or a blend of branched chain esters known as Crodamol CAP may be used. These may be used alone or in combination depending on the properties required. Alternatively, high melting point lipids such as white soft paraffin and/or liquid paraffin or other mineral oils can be used.

In practice, a compound/pharmaceutical composition of the present invention may be administered in a suitable formulation to humans and animals by topical or systemic administration, including oral, inhalational, rectal, nasal, buccal, sublingual, vaginal, colonic, parenteral (including subcutaneous, intramuscular, intravenous, intradermal, intrathecal and epidural), intracisternal and intraperitoneal. It will be appreciated that the preferred route may vary with for example the condition of the recipient.

“Pharmaceutically acceptable dosage forms” refers to dosage forms of the compound of the invention, and includes, for example, tablets, dragées, powders, elixirs, syrups, liquid preparations, including suspensions, sprays, inhalants tablets, lozenges, emulsions, solutions, granules, capsules and suppositories, as well as liquid preparations for injections, including liposome preparations. Techniques and formulations generally may be found in Remington's Pharmaceutical Sciences, Mack Publishing Co., Easton, Pa., latest edition.

“Formulations suitable for oral administration” may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tables may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compounds moistened with an inert liquid diluent. The tablets may optionally be coated or scored and may be formulated so as to provide slow or controlled release of the active ingredient therein.

Solid compositions for rectal administration include suppositories formulated in accordance with known methods and containing at least one compound of the invention.

If desired, and for more effective distribution, the compounds can be microencapsulated in, or attached to, a slow release or targeted delivery systems such as a biocompatible, biodegradable polymer matrices (e.g., poly(d,l-lactide co-glycolide)), liposomes, and microspheres and subcutaneously or intramuscularly injected by a technique called subcutaneous or intramuscular depot to provide continuous slow release of the compound(s) for a period of 2 weeks or longer. The compounds may be sterilized, for example, by filtration through a bacteria retaining filter, or by incorporating sterilizing agents in the form of sterile solid compositions which can be dissolved in sterile water, or some other sterile injectable medium immediately before use.

“Formulations suitable for oral administration” means formulations which are in a form suitable to be administered orally to a patient. The formulations may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

“Formulations suitable for parenteral administration” means formulations that are in a form suitable to be administered parenterally to a patient. The formulations are sterile and include emulsions, suspensions, aqueous and non-aqueous injection solutions, which may contain suspending agents and thickening agents and anti-oxidants, buffers, bacteriostats and solutes which render the formulation isotonic, and have a suitably adjusted pH, with the blood of the intended recipient.

“Formulations suitable for systemic administration” means formulations that are in a form suitable to be administered systemically to a patient. The formulation is preferably administered by injection, including transmuscular, intravenous, intraperitoneal, and subcutaneous. For injection, the compounds of the invention are formulated in liquid solutions, in particular in physiologically compatible buffers such as Hank's solution or Ringer's solution. In addition, the compounds may be formulated in solid form and redissolved or suspended immediately prior to use. Lyophilized forms are also included. Systematic administration also can be by transmucosal or transdermal means, or the compounds can be administered orally. For transmucosal or transdermal administration, penetrants appropriate to the barrier to be permeated are used in the formulation. Such penetrants are generally known in the art, and include, for example, bile salts and fusidic acid derivatives for transmucosal administration. In addition, detergents may be used to facilitate permeation. Transmucosal administration may be through use of nasal sprays, for example, or suppositories. For oral administration, the compounds are formulated into conventional oral administration forms such as capsules, tablets, and tonics.

“Formulations suitable for topical administration” means formulations that are in a form suitable to be administered topically to a patient. The formulation may be presented as a topical ointment, salves, powders, sprays and inhalants, gels (water or alcohol based), creams, as is generally known in the art, or incorporated into a matrix base for application in a patch, which would allow a controlled release of compound through the transdermal barrier. When formulated in an ointment, the active ingredients may be employed with either a paraffinic or a water-miscible ointment base. Alternatively, the active ingredients may be formulated in a cream with an oil-in-water cream base. Formulations suitable for topical administration in the eye include eye drops wherein the active ingredient is dissolved or suspended in a suitable carrier, especially an aqueous solvent for the active ingredient. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

“Solid dosage form” means the dosage form of the compound of the invention is solid form, for example capsules, tablets, pills, powders, dragées or granules. In such solid dosage forms, the compound of the invention is admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alginates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example paraffin, (f) absorption accelerators, as for example, quaternary ammonium compounds, (g) wetting agents, as for example, cetyl alcohol and glycerol monostearate, (h) adsorbents, as for example, kaolin and bentonite, (i) lubricants, as for example, talc, calcium stearate, magnesium stearate, solid polyethylene glycols, sodium lauryl sulfate, (j) opacifying agents, (k) buffering agents, and agents which release the compound(s) of the invention in a certain part of the intestinal tract in a delayed manner.

Actual dosage levels of active ingredient(s) in the compositions of the invention may be varied so as to obtain an amount of active ingredient(s) that is (are) effective to obtain a desired therapeutic response for a particular composition and method of administration for a patient. A selected dosage level for any particular patient therefore depends upon a variety of factors including the desired therapeutic effect, on the route of administration, on the desired duration of treatment, the etiology and severity of the disease, the patient's condition, weight, sex, diet and age, the type and potency of each active ingredient, rates of absorption, metabolism and/or excretion and other factors.

Total daily dose of the compounds of this invention administered to a patient in single or divided doses may be in amounts, for example, of from about 0.001 to about 100 mg/kg body weight daily and preferably 0.01 to 10 mg/kg/day. For example, in an adult, the doses are generally from about 0.01 to about 100, preferably about 0.01 to about 10, mg/kg body weight per day by inhalation, from about 0.01 to about 100, preferably 0.1 to 70, more especially 0.5 to 10, mg/kg body weight per day by oral administration, and from about 0.01 to about 50, preferably 0.01 to 10, mg/kg body weight per day by intravenous administration. The percentage of active ingredient in a composition may be varied, though it should constitute a proportion such that a suitable dosage shall be obtained. Dosage unit compositions may contain such amounts of such submultiples thereof as may be used to make up the daily dose. Obviously, several unit dosage forms may be administered at about the same time. A dosage may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. It goes without saying that, for other patients, it will be necessary to prescribe not more than one or two doses per day.

The formulations can be prepared in unit dosage form by any of the methods well known in the art of pharmacy. Such methods include the step of bringing into association the active ingredient with the carrier that constitutes one or more accessory ingredients. In general the formulations are prepared by uniformly and intimately bringing into association the active ingredient with liquid carriers or finely divided solid carriers or both, and then, if necessary, shaping the product.

The formulations may be presented in unit-dose or multi-dose containers, for example sealed ampoules and vials with elastomeric stoppers, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Compounds within the scope of the present invention exhibit marked pharmacological activities according to tests described in the literature and herewith, which tests results are believed to correlate to pharmacological activity in humans and other mammals.

The chemical reactions described in the references cited above are generally disclosed in terms of their broadest application to the preparation of the compounds of this invention.

Occasionally, the reactions may not be applicable as described to each compound included within the scope of compounds disclosed herein. The compounds for which this occurs will be readily recognized by those skilled in the art. In all such cases, either the reactions can be successfully performed by conventional modifications known to those skilled in the art, e.g., by appropriate protection of interfering groups, by changing to alternative conventional reagents, by routine modification of reaction conditions, and the like, or other reactions disclosed herein or otherwise conventional will be applicable to the preparation of the corresponding compounds of this invention. In all preparative methods, all starting materials are known or readily preparable from known starting materials.

The regimen for treating a patient suffering from a macular degeneration with the compounds and/or compositions of the present invention is selected in accordance with a variety of factors, including the age, weight, sex, diet, and medical condition of the patient, the severity of the infection, the route of administration, pharmacological considerations such as the activity, efficacy, pharmacokinetic, and toxicology profiles of the particular compounds employed, and whether a drug delivery system is utilized. Administration of the drug combinations disclosed herein should generally be continued over a period until acceptable, indicating that has been controlled or eradicated. Patients undergoing treatment with the drug combinations disclosed herein can be routinely monitored by fundoscopy to determine the effectiveness of therapy. Continuous analysis of the data obtained by these methods permits modification of the treatment regimen during therapy so that optimal amounts of each component in the combination are administered, and so that the duration of treatment can be determined as well. Thus, the treatment regimen/dosing schedule can be rationally modified over the course of therapy so that the lowest amounts of each of the compounds used in combination which together exhibit satisfactory effectiveness are administered, and so that administration of such compounds in combination is continued only so long as is necessary to successfully treat the macular degeneration, diabetic retinopathy, or diabetic macular edema.

An aspect of the present invention encompasses the use of combinations of anti-VEGF inhibitors and compounds having Syk activity as described above to treat or prevent macular degeneration where one or more of these compounds is present in a pharmaceutically effective amount, and the other(s) is(are) present in a subclinical pharmaceutically effective or an effective amount(s) owing to their additive or synergistic effects. As used herein, the term “additive effect” describes the combined effect of two (or more) pharmaceutically active agents that is equal to the sum of the effect of each agent given alone. A “synergistic effect” is one in which the combined effect of two (or more) pharmaceutically active agents is greater than the sum of the effect of each agent given alone.

The present invention may be embodied in other specific forms without departing from the spirit or essential attributes thereof.

What is claimed is:

1. A method of treating a patient for an ocular related disorder, wherein the method comprises administering to the patient a pharmaceutically effective amount of a compound of formula I, 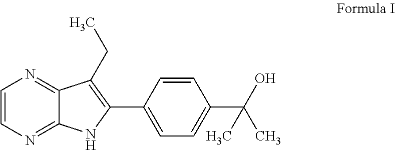
or a pharmaceutically acceptable salt or prodrug thereof.

2. The method of claim 1, wherein the ocular related disorder is ocular neovascularization.

3. The method of claim 2, wherein the ocular related disorder is ocular neovascularization of the retina.

4. The method of claim 1, wherein the ocular related disorder is selected from: age related macular degeneration, diabetic retinopathy, and diabetic macular edema.

5. The method of claim 4, wherein the ocular disorder is age related macular degeneration.

6. The method of claim 4, wherein the ocular disorder is diabetic retinopathy.

7. The method of claim 4, wherein the ocular disorder is diabetic macular edema.

8. The method of claim 1, wherein the compound of formula I is administered to a patient who is concurrently being treated with ranizumab.

9. A pharmaceutical composition for treating macular degeneration, diabetic retinopathy, or diabetic macular edema, comprising a compound of Formula I: 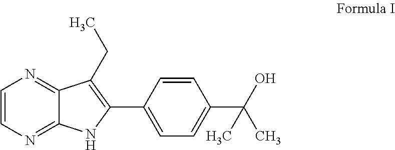
or a pharmaceutically acceptable salt or prodrug thereof.

10. A method of treating macular degeneration, diabetic retinopathy, or diabetic macular edema, comprising: administering to a patient in need thereof a pharmaceutically effective amount of a Syk kinase inhibitor.
